(19) United States
(12) Patent Application Publication
(10) Pub. No.: US 2003/0108615 A1
(43) Pub. Date: Jun. 12, 2003

(54) Pharmaceutical formulation comprising carrier beads coated with a layer of valaciclovir

(76) Inventors: Koho Kawamura, Research Triangle Park, NC (US); Yasuhiro Mishima, Research Triangle Park, NC (US); Kiyoshi Ohtsuka, Research Triangle Park, NC (US); Nobuya Sugibayashi, Research Triangle Park, NC (US); Keiichi Sugisawa, Research Triangle Park, NC (US); Hitomi Yanagihara, Research Triangle Park, NC (US); Makio Yasuda, Research Triangle Park, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/258,506

(22) PCT Filed: Apr. 27, 2001
(86) PCT No.: PCT/JP01/03738

(30) Foreign Application Priority Data
Apr. 28, 2000 (GB) 0010446.3

(51) Int. Cl. A61K 31/522; A61K 9/16; A61K 9/50

(52) U.S. Cl. 424/490; 514/263.38

(57) ABSTRACT

A pharmaceutical formulation for valaciclovir comprising carrier beads coated with one or more layers of pharmaceutically acceptable substances, wherein, at least one layer comprises valaciclovir, or a pharmaceutically acceptable derivative thereof, as the active ingredient. There is also provided a process for the preparation of such a formulation.

The present invention relates to a pharmaceutical formulation of the antiviral drug valaciclovir, or a pharmaceutically acceptable derivative thereof, useful in the treatment of disease in mammals, including humans. The invention also relates to a process for the preparation of such a formulation.

The compound 9-[(2-hydroxyethoxy)methyl]guanine, otherwise known as acyclovir possesses potent antiviral activity and is widely used in the treatment and prophylaxis of viral infections in humans, particularly infections caused by the herpes group of viruses (see, for example, Schaeffer et al. Nature, 272, 583-585 (1978), UK Patent No. 1523856, U.S. Pat. No. 4,199,574). However, acyclovir is poorly absorbed from the gastrointestinal tract upon oral administration and this low bioavailability means that multiple high doses of oral drug may need to be administered, especially for the treatment of less sensitive viruses or infections in order to achieve and maintain effective anti-viral levels in the plasma.

The L-valine ester of acyclovir [2-(2-amino-1,6-dihydro-6-oxo-purin-9-yl)methoxy]ethyl L-valinate (hereinafter referred to as valaciclovir) has been shown to possess much improved bioavailability whilst retaining the anti-viral properties of acyclovir. A prefered form of this compound is its hydrochloride salt which is herein referred to as valaciclovir hydrochloride. Valaciclovir and its salts including the hydrochloride salt are disclosed in U.S. Pat. No. 4,957,924, European Patent No. 0308065 and Beauchamp et al, Antiviral Chemistry and Chemotherapy, 3(3), 154-164 (1992).

Valaciclovir and methods for its preparation are disclosed in inter alia, European Patent No. 0308065 and PCT publication WO 96/22082, both incorporated herein by reference. European Patent No. 0308065 generically discloses formulations for oral administration including granules, however no formulations for oral granules are specifically disclosed therein. WO 96/22082, discloses the formulation of valaciclovir into tablets. A further PCT publication, WO 96/32097, discloses sustained release compositions for non-amorphous active ingredients such as valaciclovir.

Valaciclovir has proven difficult to formulate. Certain difficulties of formulating valaciclovir are described in WO 96/22082. For example, WO 96/22082 explains that it is difficult to obtain a tablet of sufficient hardness and friability for pharmaceutical handling and for film coating. One problem is that valaciclovir has “adhesive” properties in that it can stick to the tablet dies and therefore needs to be efficiently lubricated. The formulation of granules of valaciclovir was also found to be problematic due to the adhesive properties of valaciclovir and also to problems relating to the pH dependent dissolution rate and discolouration of the granules. Granules of valaciclovir itself tend to be fragile and have a low coating efficacy. Furthermore, there is a need to mask the bitter taste of valaciclovir. It is therefore an object of the invention to provide a suitable granule formulation for valaciclovir.

We have found that pharmaceutical formulations of the present invention are particularly suitable for formulating oral granules comprising valaciclovir, or a pharmaceutically acceptable derivative thereof. Oral granules have certain advantages also associated with tablets, i.e. they can easily be stored and are stable over long periods, but oral granules are preferred in certain countries as they represent advantages over tablets in that the dose can be more easily manipulated depending on the body weight of the patient. They may also be more easily administered for those patients who have difficulty swallowing tablets, e.g. granules are convenient for oral administration to children and the elderly.

The present invention accordingly provides a pharmaceutical formulation, comprising carrier beads coated with one or more layers of pharmaceutically acceptable substances, wherein, at least one layer is a drug coat layer comprising valaciclovir, or a pharmaceutically acceptable derivative thereof, as the active ingredient.

As used herein “pharmaceutically acceptable derivative” includes any pharmaceutically acceptable salt, ester, or salt of such ester of valaciclovir which, upon administration to the recipient, is capable of providing (directly or indirectly) valaciclovir or an antivirally active metabolite or residue thereof. Pharmaceutically acceptable salts of valaciclovir are preferably acid addition salts derived from an appropriate acid, e.g. hydrochloric, sulphuric, phosphoric, maleic, fumaric, citric, tartaric, lactic, acetic or p-toluenesulphonic acid. A particularly preferred salt is the hydrochloride salt of valaciclovir.

Valaciclovir or pharmaceutically acceptable derivatives thereof are hereinafter referred to generally as the ‘active ingredient’.

Suitable formulations contain 1 to 90% by weight of active ingredient. Preferred formulations contain from 25 to 75% by weight of active ingredient, desirably from 50 to 60%.

In the present invention the active ingredient is provided on carrier beads. The use of a solid core (carrier beads) makes it possible to achieve excellent coating efficacy due to the uniform shape and size of the solid core. A carrier bead comprises a pharmaceutically acceptable substance other than the active ingredient which provides a surface onto which one or more layers of pharmaceutically acceptable substances, at least one of which comprises the active ingredient, may be coated. Suitable carrier beads may comprise cellulose (e.g. Celluphia CP305, Celluphia CP203), non-pareils (carrier beads made of sucrose) (e.g. Freund Industry), lactose, starch or microcrystalline cellulose. Preferably the carrier beads comprise cellulose (e.g. Celluphia CP305, Asahi Chemical Industry). Celluphia CP305 is pure cellulose. Preferably the carrier beads are present in amounts of 1 to 50% by weight of the total formulation, more preferably 20 to 40%. Preferably the range of particle sizes of the solid core is 300 to 500 μm, more preferably the average particle size of the solid core is 400 μm in diameter.

In a preferred aspect of the invention, the drug coat layer further comprises a component which acts as a binder. Suitable binders include cellulose (e.g. Hydroxy Propyl Cellulose (HPC-L, Nippon Soda), Hydroxy Propyl Methyl Cellulose (HPMC)), starch and polyvinylpyrrolidone (e.g. PVP K30, PVP K90). A particularly preferred binder, providing strong binding effects with valaciclovir and good coating efficiency, is polyvinylpyrrolidone (PVP). PVP may be PVP K30 (a linear polymer of 1-vinyl-2-pyrrolidone having an average molecular weight of about 40000) or PVP K90 (a linear polymer of 1-vinyl-2-pyrrolidone having an average molecular weight of about 1200000) (e.g. ISP, BASF). Particularly preferred is polyvinylpyrrolidone grade K30. Preferably the binder is present in amounts of 1 to 30% by weight of the total formulation, more preferably 1 to 10%.

As valaciclovir has a particularly unpleasant taste, the formulation of the present invention preferably further comprises a mask coat layer. Preferably the mask coat layer comprises a polymer, a lubricant and a binder/dissolution enhancer. Suitable polymers include Eudragit (Methacrylic acid copolymer as described in Japanese Pharmaceutical Excipients, 1998 (e.g. NE30D, L30D55, L10055, L100, E100, S100, RS100L, RS30DL, RS100, RS30D)); Cellulose (HPC, HPMC, Hydroxy Propyl Methyl Cellulose Phthalate (HPMCP), Ethyl Cellulose (EC), Carboxy Methyl Ethyl Cellulose. (CMEC), Cellulose Acetate Phthalate (CAP), Hydroxy Ethyl Cellulose (HEC)); wax (glyceryl monostearate etc.); gelatin and shellac. A preferred polymer is a methacrylic acid copolymer e.g. Eudragit, most preferably Eudragit NE30D. Eudragit NE30D offers the advantage of pH-independent solubility. Preferably the polymer is present in amounts 1 to 30% by weight of the total formulation, more preferably 1 to 10%. Suitable lubricants include talc, kaolin, magnesium stearate and stearic acid. A preferred lubricant is talc. Preferably the lubricant is present in amounts 1 to 30% by weight of the total formulation, more preferably 1 to 10%. Suitable binder/dissolution enhancers include HPC-L, PVP, starch, HPMC Polyethylene glycol 6000 (PEG 6000), Polysorbate 80, Triacetin, Titanium Dioxide and Sodium Lauryl Sulphate. Preferably the binder/dissolution enhancer is HPC-L. Preferably the binder/dissolution enhancer is present in amounts 0.1 to 20% by weight of the total formulation, more preferably 0.1 to 5%.

Preferably a terminal blend is applied to the coated carrier beads to make final granules. The terminal blend helps to prevent the agglomeration and adhesion of the final granules which is associated with the adhesive property of valaciclovir. The terminal blend comprises one or more lubricants. Suitable lubricants are talc, aerosil, kaolin, magnesium stearate and stearic acid. Preferred lubricants are talc and aerosil. Talc is a purified, hydrated, magnesium silicate, approximating to the formula Mg₆(Si₂O₅)₄(OH)₄. A suitable talc is CROWN TALC KYOKU PP (Matsumura Industrial). Preferably the terminal blend comprises 0.1 to 20% by weight of the total formulation, more preferably 0.1 to 10%.

The formulations of the invention may, if desired, further include one or more pharmaceutically acceptable carriers or excipients. All such excipients must be “pharmaceutically acceptable” in the sense of being compatible with the other ingredients of the formulation and not injurious to the patient. Pharmaceutically acceptable excipients may include colours, flavours e.g. menthol, sweeteners e.g. mannitol, and other excipients known to those skilled in the art.

A preferred formulation of the invention comprises:

(a) carrier beads, comprising from 1 to 50% by weight of the total formulation;

(b) a drug coat layer comprising from 1 to 90% by weight of the total formulation comprising valaciclovir, or a pharmaceutically acceptable derivative thereof, and from 1 to 30% by weight of the total formulation of a component which acts as a binder;

(c) a mask coat layer comprising from 1 to 30% by weight of the total formulation of a polymer, from 1 to 30% by weight of the total formulation of a component which acts as a lubricant, and from 0.1 to 20% by weight of the total formulation of a component which acts as a binder/dissolution enhancer; and

(d) a terminal blend comprising from 0.1 to 20% by weight of the total formulation of a component which acts as a lubricant.

An especially preferred formulation of the invention comprises:

(a) carrier beads, comprising from 20 to 40% by weight of the total formulation;

(b) a drug coat layer comprising from 25 to 75% by weight of the total formulation comprising valaciclovir, or a pharmaceutically acceptable derivative thereof, and from 1 to 10% by weight of the total formulation of a component which acts as a binder;

(c) a mask coat layer comprising from 1 to 10% by weight of the total formulation of a polymer, from 1 to 10% by weight of the total formulation of a component which acts as a lubricant and from 0.1 to 5% by weight of the total formulation of a component which acts as a binder/dissolution enhancer; and

(d) a terminal blend comprising from 0.1 to 10% by weight of the total formulation of a component which acts as a lubricant.

A preferred specific formulation of the invention comprises:

(a) cellulose carrier beads, comprising about 30.6% by weight of the total formulation;

(b) a drug coat layer comprising about 55.6% by weight of the total formulation of valaciclovir hydrochloride, and about 4.2% by weight of the total formulation of polyvinylpyrrolidone as a binder;

(c) a mask coat layer comprising about 4.11% by weight of the total formulation of methacrylic acid copolymer, about 4.11% by weight of the total formulation of talc as a lubricant, and about 0.89% by weight of the total formulation of hydroxypropylcellulose as a binder/dissolution enhancer; and

(d) a terminal blend comprising about 0.25% by weight of the total formulation of talc and 0.25% by weight of the total formulation of aerosil which act as lubricants.

It is to be understood that the present invention covers all combinations of particular and preferred groups described hereinabove.

To meet the standard on granules defined in the Japanese Pharmacopoeia, it is preferable that:

(i) no granules have a particle size greater than 1700 μm.

(ii) no more than 5% by weight of the granules have a particle size greater than 1400 μm. Preferably no more than 4% by weight of the granules have a particle size greater than 1400 μm, for example, no more than 3% by weight. Desirably, less than 0.5% of the granules have a particle size greater than 1400 μm.

(iii) no more than 15% by weight of the granules have a particle size smaller than 355 μm. Preferably no more than 10% by weight of the granules have a particle size smaller than 355 μm, for example, no more than 5% by weight. Desirably, less than 0.5% of the granules have a particle size smaller than 355 μm. Preferably up to 98 to 100% by weight of the granules of the invention have such a particle size.

Particle size is determined by the Particle Size Distribution Test for Granules, The Japanese Pharmacopoeia, Thirteenth Edition 1996.

Formulations of the present invention preferably have the following properties:

(i) no granules have a particle size greater than 1700 μm.

(ii) at least 95% by weight of the granules have a particle size of from 355 μm to 1400 μm.

(iii) no more than 0.5% by weight of the granules have a particle size smaller than 355 μm.

Preferably the range of particle sizes for the finished granules is 355 to 850 μm, more preferably the average particle size of the finished granules is 800 μm.

The present invention provides a pharmaceutical formulation for use in medical therapy, e.g. in the treatment of a viral disease in an animal, e.g. a mammal such as a human. The compound is especially useful for the treatment of diseases caused by various DNA viruses, such as herpes infections, for example, herpes simplex 1 and 2, varicella zoster, cytomagalovirus, Epstein-Barr viruses or human herpes virus-6 (HHV-6) as well as diseases caused by hepatitis B. The active compound can also be used for the treatment of papilloma or wart virus infections, and may furthermore be administered in combination with other therapeutic agents, for example, with zidovudine, to treat retroviral associated infections in particular HIV infections.

In addition to its use in human medical therapy, the active compound can be administered to other animals for treatment of viral diseases, e.g. to other mammals. In particular, the compound is active against herpes simplex virus which causes herpetic keratitis in rabbits, herpetic encephalitis in mice, and cutaneous herpes in guinea pigs.

For each of the above-indicated utilities and indications the amount required of the active ingredient will depend on a number of factors including the severity of the condition to be treated and the identity of the recipient and will ultimately be at the discretion of the attendant physician or veterinarian. In general, however, for each of these utilities and indications, a suitable effective dose will be in the range 1 to 150 mg per kilogram bodyweight of recipient per day, preferably in the range 5 to 120 mg per kilogram bodyweight per day (Unless otherwise indicated, all weights of the active ingredient are calculated with respect to the free base valaciclovir). The desired dose is preferably presented as one, two, three or four or more sub-doses administered at appropriate intervals throught the day. These sub-doses may be administered in unit dosage forms, for example, containing about 50 to 2000 mg, preferably about 250, 500, 1000 or 2000 mg of active ingredient per unit dose form, most preferably about 500 mg of active ingredient per unit dose form.

The following dosage regimes are given for guidance: treatment of herpes simplex virus types 1 and 2 infection: total daily dose of about 1 or 2 g administered at 500 mg twice a day or 1 g twice a day for 5 to 10 days; suppression of herpes simplex virus types 1 and 2 infections: total daily dose about 250 mg to 1 g for about one to ten years (depending on the patient, age or length of treatment); treatment of varicella zoster virus infections (for example shingles): daily dose about 3 g administered at 1 g three times a day for seven days; supression of cytomegalovirus infections: total daily dose about 8 g administered at 2 g four times a day. For transplant patients this daily dose is administered for three to six months for the period at risk; and for HIV positive patients said daily dose is administered as usually indicated for improving quality of life, for example, for two years or more.

Early results now indicate that valaciclovir can be used in the effective suppression of recurrent genital herpes at a once daily dose of from about 200 mg to about 1000 mg for an effective treatment period. The most likely daily dosages are 250 mg, 500 mg or 1000 mg. For example, a valaciclovir dose of 500 mg may be provided in 1 g of granules.

The term “treatment” and derivatives such as “treating” as used herein includes both treatment and prophylaxis.

A further aspect of the invention provides a process for preparing granules comprising coating a carrier bead with a drug coat layer comprising valaciclovir, or a pharmaceutically acceptable derivative thereof, as the active ingredient and, where appropriate, further coating with a mask coat layer and/or terminal blend.

Methods for making valaciclovir are disclosed in European Patent No. 0308065. Valaciclovir hydrochloride (anhydrous crystalline form) can be made as described in WO 96/22082, the contents of which are incorporated herein by reference.

Carrier beads may be granulated into beads using an aqueous solution or organic solvents such as ethanol or IPA. Preferably the carrier beads are granulated using an aqueous solution, more preferably the aqueous solution is water. Celluphia round beads are granules of microcrystalline cellulose powder, granulated with water (Asahi Chemical Industry). Granulation is carried out using a suitable granulator, as known to a person skilled in the art. The resulting granules may be passed through a sieve to select particle size. Carrier beads of an average particle size 300 to 500 μm are preferred, more preferably the average particle size is 400 μm in diameter.

Layers of pharmaceutically acceptable substances may be applied to carrier beads using a non-aqueous system with ethanol or IPA as a solvent, or alternatively using an aqueous system, e.g. purified water. The aqueous system offers the advantage that the process may be safer and cheaper. A preferred feature of the invention is therefore the use of water as a solvent to form a layer of the active ingredient. The water used is preferably purified water, purified that is according to the standards of the Japanese Pharmacopoeia.

A drug coat layer may be prepared by first heating a solvent, e.g. purified water, suitably to 65° C., and dissolving the active ingredient and binder in the solvent. The solution is then cooled to room temperature and filtered. The obtained aqueous suspension (the active ingredient and binder) is sprayed onto carrier beads and the coated carrier beads are dried in a rotated fluid bed granulator.

A mask coat layer may be prepared by mixing the polymer, lubricant and binder/dissolution enhancer and a solvent, e.g. purified water, to form a suspension and filtering the suspension. The mask coat suspension is sprayed onto drug coated beads and the coated carrier beads are dried in a rotated fluid bed granulator.

The dried granules are subjected to sieving to ensure that the appropriate particle size requirements are met. Preferably the range of particle sizes for the finished granules is 355 to 850 μm. More preferably, the average particle size of the finished granules is 800 μm.

Finally, the granules are blended with lubricants in a suitable blender as known to a person skilled in the art.

The resulting granules may be taken directly or incorporated into pharmaceutical compositions for oral administration. The granules may be administered in an inert liquid vehicle so as to form a drench, or in a suspension with a water or oil base. For example, the granules may be administered in a syrup. Preferably, the granules are taken directly.

The formulation may be introduced into a container which is then closed. The container may be sealed. It may be a single-dose or multi-dose container. The container may be bottles, jars, bags or sachets. Sachets, especially foil sachets (foil-foil blisters), are particularly suitable for single dose packaging. Bottles, particularly high density polyethylene (HDPE) bottles are particularly suitable for multi-dose packaging.

The following examples illustrate aspects of this invention but should not be construed as limiting the scope of the invention in any way.

| Process | Ingredients | Amount (%) |
|---|---|---|
| Drug Coat | Carrier beads: Cellulose (Celluphia CP305) | 30.6 |
| | Valaciclovir HCl | 55.6 |
| | (as free base) | (50.0) |
| | Binder: polyvinylpyrrolidone (PVP K30) | 4.2 |
| Mask Coat | Polymer: Methacrylic acid copolymer | 4.11 |
| | (Eudragit NE30D) | (as solid) |
| | Lubricant: Talc | 4.11 |
| | Binder/dissolution enhancer: Hydroxypropyl | 0.89 |
| | cellulose (HPC-L) | |
| Terminal | Lubricants: Talc | 0.25 |
| Blend | Aerosil | 0.25 |
| Total | | 100 |

1) Drug Coating

Purified water was heated to 65° C. and PVP K30 and valaciclovir HCl were added to the water and dissolved in a stainless mixing tank with jacket heater. Then, the solution was cooled to room temperature. The obtained aqueous suspension was filtered and sprayed onto Celluphia CP305, and dried in a rotated fluid bed granulator.

2) Mask Coating

HPC-L was dissolved in purified water and Talc and Eudragit NE30D were added to the solution, and dispersed in a stainless mixing tank. The coating suspension was filtered and sprayed onto drug coated beads, and dried in the same machine.

3) Terminal Blend

The mask coated beads, Talc and Aerosil were blended in a V-shaped blender.

4) Packaging

The obtained final granules (beads) were filled in bottles, jars, bags or sachets. Appearance: White Round Granules

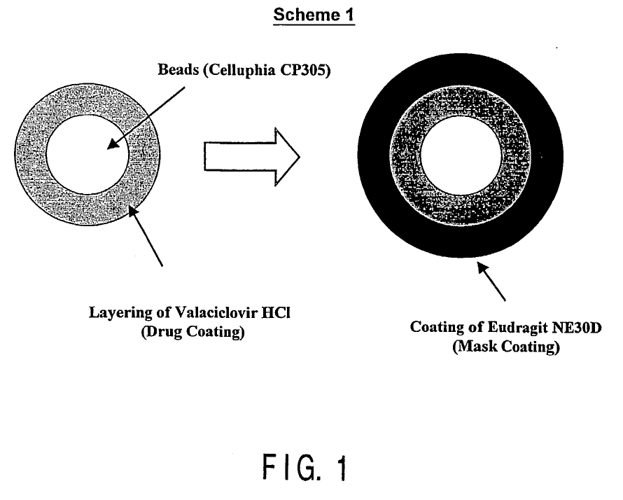

FIG. 1 is

Scheme 1 shows the preparation of carrier beads of Celluphia CP305 coated with a drug coat layer comprising valaciclovir HCl to which a mask coat layer comprising Eudragit NE30D is applied.

### Example 2

| | | | |
|---|---|---|---|
| Carrier Beads | Sucrose | Nonpareils #32-42 | 20-40% |
| | | Nonpareils #20-40 | |
| | | Nonpareils #20-24 | |
| | Lactose | Lactose#50 | |
| | | Lactose #30-50 | |
| | Microcrystalline | Celluphia CP203 | |
| | cellulose | | |
| Binders in the drug | Cellulose | HPC-L | 1-30% |
| coat layer | Starch | Corn Starch | |
| | Povidone | K90 | |
| Polymers in the | Eudragit | L100 | 1-25% |
| mask coat layer | | L30D55 | |
| | | RS30D | |
| | Cellulose | Ethyl Cellulose | |
| Binder/dissolution | Cellulose | HPMC | 0.5-10% |
| enhancers | Others | PEG6000 | |
| in the mask coat | | Polysorbate 80 | |
| layer | | Triacetin | |
| | | Titanium Dioxide | |
| | | SLS | |
| | | TEC | |

1) Drug Coating

As alternative carrier beads, Nonpareils (#32-42, #20-40, #20-24), Lactose (#50, #30-50) or Celluphia CP203 may be used. The amount of these ingredients are 20 to 40% by weight of the total formulation. HPC-L, Corn Starch or PVP K90 can be used as a binder and the amount is 1 to 30% by weight of the total formulation.

2) Mask Coating

As alternative mask coat polymers, Eudragit (L100, L30D55, RS30D) or Ethyl Cellulose may be used. The amount of these ingredients are 1 to 30%, e.g. 1 to 25%, by weight of the total formulation. Other binder/dissolution enhancers (HPMC, Polyethyleneglycol 6000 (PEG6000), Polysorbate80, Triacetin, Titanium Dioxide, Sodium Lauryl Sulphate (SLS) or Triethyl citrate (TEC)) can be used and the amount is 0.1 to 20%, e.g. 0.5 to 10%, by weight of the total formulation.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any novel feature or combination of features described herein. This may take the form of product, composition, process or use claims and may include, by way of example and without limitation, one or more of the following claims.

1. A pharmaceutical formulation comprising carrier beads coated with one or more layers of pharmaceutically acceptable substances, wherein, at least one layer is a drug coat layer comprising valaciclovir, or a pharmaceutically acceptable derivative thereof, as the active ingredient.

2. A pharmaceutical formulation according to claim 1 wherein the drug coat layer further comprises a component which acts as a binder.

3. A pharmaceutical formulation according to claim 1 or 2 further comprising a mask coat layer.

4. A pharmaceutical formulation according to claim 3 wherein the mask coat layer comprises a polymer, a component which acts as a lubricant and a component which acts as a binder/dissolution enhancer.

5. A pharmaceutical formulation as claimed in any one of claims 1-4 further comprising a terminal blend.

6. A pharmaceutical formulation as claimed in any one of claims 1-5, which comprises:
(a) carrier beads, comprising from 1 to 50% by weight of the total formulation;
(b) a drug coat layer comprising from 1 to 90% by weight of the total formulation comprising valaciclovir, or a pharmaceutically acceptable derivative thereof, and from 1 to 30% by weight of the total formulation of a component which acts as a binder;
(c) a mask coat layer comprising from 1 to 30% by weight of the total formulation of a polymer, from 1 to 30% by weight of the total formulation of a component which acts as a lubricant and from 0.1 to 20% by weight of the total formulation of a component which acts as a binder/dissolution enhancer; and
(d) a terminal blend comprising from 0.1 to 20% by weight of the total formulation of a component which acts as a lubricant.

7. A pharmaceutical formulation as claimed in claim 6, which comprises:
(a) carrier beads, comprising from 20 to 40% by weight of the total formulation;
(b) a drug coat layer comprising from 25 to 75% by weight of the total formulation comprising valaciclovir, or a pharmaceutically acceptable derivative thereof, and from 1 to 10% by weight of the total formulation of a component which acts as a binder;
(c) a mask coat layer comprising from 1 to 10% by weight of the total formulation of a polymer, from 1 to 10% by weight of the total formulation of a component which acts as a lubricant, and from 0.1 to 5% by weight of the total formulation of a component which acts as a binder/dissolution enhancer; and
(d) a terminal blend comprising from 0.1 to 10% by weight of the total formulation of a component which acts as a lubricant.

8. A pharmaceutical formulation as claimed in claim 7, which comprises:
(a) cellulose carrier beads, comprising about 30.6% by weight of the total formulation;
(b) a drug coat layer comprising about 55.6% by weight of the total formulation of valaciclovir hydrochloride, and about 4.2% by weight of the total formulation of polyvinylpyrrolidone as a binder;
(c) a mask coat layer comprising about 4.11% by weight of the total formulation of methacrylic acid copolymer, about 4.11% by weight of the total formulation of talc as a lubricant, and about 0.89% by weight of the total formulation of hydroxypropylcellulose as a binder/dissolution enhancer; and
(d) a terminal blend comprising about 0.25% by weight of the total formulation of talc and 0.25% by weight of the total formulation of aerosil which act as a lubricant.

9. A pharmaceutical formulation as claimed in any one of claims 1-8 together with one or more pharmaceutically acceptable carriers or excipients.

10. Use of a pharmaceutical formulation as claimed in any one of claims 1-9 in medical therapy.

11. Use of a pharmaceutical formulation as claimed in any one of claims 1-9 for the treatment of viral infections.

12. Use of a pharmaceutical formulation as claimed in any one of claims 1-9 in the manufacture of a medicament for the treatment of viral infections.

13. A method of treating a viral infection comprising administering a pharmaceutical formulation as claimed in any one of claims 1-9.

14. The use or method as claimed in claim 11, 12 or 13 wherein the viral infection is caused by a member of the herpes family of viruses.

15. A process for preparing a pharmaceutical formulation as claimed in any one of claims 1-9 comprising coating a carrier bead with a drug coat layer comprising valaciclovir, or a pharmaceutically acceptable derivative thereof.

16. A process for preparing a pharmaceutical formulation as claimed in claim 15 wherein the drug coat layer is applied to the carrier bead using an aqueous solvent.
